(19) United States
(12) Patent Application Publication
(10) Pub. No.: US 2022/0254182 A1
(43) Pub. Date: Aug. 11, 2022

(54) OPTICAL TECHNIQUE FOR ANALYZING INSECTS, SHRIMP AND FISH

(71) Applicant: DIPTERA.AI LTD., Jerusalem (IL)

(72) Inventors: Ariel LIVNE, Jerusalem (IL); Elly ORDAN, Jerusalem (IL)

(21) Appl. No.: 17/679,417

(22) Filed: Feb. 24, 2022

Related U.S. Application Data
(63) Continuation-in-part of application No. PCT/IL2020/050927, filed on Aug. 25, 2020.
Application No. PCT/IL2020/050927 is a continuation of application No. PCT/IL2021/051536, filed on Dec. 27, 2020.
(60) Provisional application No. 62/891,360, filed on Aug. 25, 2019.
(60) Provisional application No. 63/130,706, filed on Dec. 27, 2020.
(60) Provisional application No. 63/130,717, filed on Dec. 27, 2020.

(51) Int. Cl. G06V 40/10; A01K 61/95; A01K 67/033; G06V 10/764; G06V 10/82

(52) U.S. Cl. CPC G06V 40/10; A01K 61/95; G06V 10/145; G06V 10/764; G06V 10/82; A01K 67/033

(57) ABSTRACT

A novel technique for automated analysis of organisms like insects, shrimp and fish. The technique comprises detection of structures and/or organs in the organisms in a flow-system. Alternatively or in addition, the technique may comprise sorting of the organisms, for example sex separation of the organisms in the flow system. The technique may comprise steps of: (a) illuminating the organisms in a detection region, (b) imaging the organisms in the detection region, with an extended depth of field that spans at least one-fourth of their thickness and from at least two different directions; (c) analyzing the images for the presence of one or more features, structures and/or organs of interest.

### FIELD OF THE INVENTION

The invention relates to detection of features, structures and/or organs in organisms like insects, shrimp and fish, more specifically to the imaging-based detection and classification.

### BACKGROUND OF THE INVENTION

Imaging of objects from two or more directions is highly important for visualizing some features that otherwise may not be seen when viewed from a single direction. It is particularly useful for analyzing developing organs in insect or fish larvae. When viewed from a single direction, as with a microscope, these organs may be obscured or hidden by fat tissue or other organs, making it difficult to determine their presence, color, morphology, size and shape. Single-perspective systems may address this problem by acquiring multiple images of the object along the optical axis. However, this method requires a significant amount of time to collect the multiple images and therefore cannot be readily applied for high throughput. Moreover, this method may further require a complicated and costly setup of adaptive optics or confocal microscopy.

Flow cytometery systems offer the ability to optically analyze a large number of samples in a rapid and automated fashion. They are typically designed for imaging microscopic objects much smaller than insect or fish larvae, such as single cells. This is usually done from a single direction and with a narrow depth of field (due to the high numerical aperture and magnification of the objectives used). A number of methods for addressing the three-dimensionality of objects in flow cytometry have been previously discussed. They are primarily intended for mapping the external 3D shape of small target objects (U.S. Pat. Nos. 6,049,381 A, 8,009,189 B2 and 8,121,388 B2). As to detecting and mapping internal structures, this currently requires that they be fluorescent (U.S. Pat. No. 5,644,388 A). This is also the limitation of the few setups designed for multi-cellular organisms (U.S. Pat. No. 6,482,652 B2 and U.S. Pat. No. 7,116,407 B2).

It is extremely rare that structures or organs of interest in living organisms fluoresce on their own and are distinguishable from their surroundings. To achieve this requires either genetic manipulations (e.g., that the organisms express fluorescent proteins of interest) or the use of organ-selective fluorescent dyes (costly; uncommon so requires development; generally toxic). However, in the case where a low-cost solution is required that does not involve genetic modifications or weaken the organism; fluorescence is not a viable option.

It would therefore be desirable to develop a non-fluorescent, flow imaging system suited for rapid, high-resolution imagery of insect or fish larvae for the binary detection of specific organs or structures of interest.

The identification and separation of male and female organisms is necessary in various agricultural and industrial aspects. For example, industrial fish farming, seeks to optimize reproduction for increased yield. This depends, in part, on the sex-ratio of males-females in the breeding tanks. In another example, some fish play an important role in genetic research, as they have short life cycles and are simple to work with and contain. In yet another example, stronger and healthier individual organisms may be required for further stages of research, breeding or farming. The identification and separation of male and female fish are required in any genetic study. Large scale sex separation can be beneficial in large scale farming because the sexes have different development times, flavor profiles and price. Current, biological sex separation methods have drawbacks such as relying on sexually dimorphic characters (e.g., size or development rate) that are subject to natural variation, requiring regular adjustment and re-calibration of the sorting systems used. They are performed on fish that already have external secondary sexual characteristics. Sex-separation methods based on the use and release of genetically modified organisms encounter regulation and public opposition.

There is still a long felt and unmet need for an efficient and cost-effective technique for rapid sex-sorting of organisms such as insects, fish and shrimp.

### GENERAL DESCRIPTION

The present invention describes an imaging flow technique (i.e., one or more methods and systems) designed to analyze organisms such as insects, fish, shrimp one-by-one, and optionally- to sort them. The organisms may be pre-adult (for example, larvae), or adult.

According to a first broad aspect of the invention, there is provided a computer implemented method for automated detection of features, structures and/or organs in organisms being insects, shrimp or fish, the method comprising:
a) providing a flow-system comprising a fluidic channel with an inlet, a detection region and at least one outlet, an electro-optical unit for monitoring the detection region, a processor;
b) ensuring passage of the organisms via a fluidic channel towards the detection region,
c) imaging an individual organism in the detection region by one or more sensors of the electro-optical module, thereby acquiring optical data on the organism,
d) transmitting the acquired optical data to the processor,
e) analyzing the obtained optical data by the processor, for detecting presence of one or more of said features, structures and/or organs of interest.

According to a second broad aspect of the invention, there is provided a system for automated detection of one or more features, structures and/or organs in organisms being insects, shrimp or fish, the system comprising:
a a fluidic channel comprising an inlet, a detection region and at least one outlet, said channel is configured to allow flowing there-though of the organisms suspended in liquid media;
b an electro-optical module for monitoring the detection region,
c a processor in communication with the electro-optical module, wherein
   said electro-optical module comprises at least one sensor configured to acquire optical data by imaging an individual organism and to transmit said optical data to the processor, and
   the processor is configured to receive, process and analyze the optical data acquired by the electro-optical module so as to detect said structures and/or organs of interest in the organism.

The proposed flow technique may be used both for pre-adult and adult organisms of the mentioned plurality including insects, shrimp and fish. It should be noted however that for insects and shrimp, the proposed technique is mainly applicable to pre-adult organisms.

In certain embodiments, it may be beneficial to determine the sex of each individual pre-adult organism, for example larva. However, as the differences at this stage are usually very subtle, and observable only in a number of structures or precursors of organs, they may be hard to detect even with high-end microscopes. Moreover, the complex interior of the larva causes light to be transmitted, refracted and/or reflected in ways that are highly dependent on the orientation of the larva with respect to any illumination sources and/or image capturing units.

To this end, this invention describes a method and a system to achieve this in a fast, reliable and reproducible manner without requiring 3D reconstruction or fluorescence labeling/expression. Specifically, the organisms (for example, larvae) flowing through the system are illuminated and imaged from a number of different directions and with extended depth of field so that the most important portion of, or the entire larva is in focus. This significantly increases the chances that the system will acquire at least one image with the needed information for each larva. Furthermore, the color of the illumination can be chosen according the level of penetration in the larvae analyzed and by how much it emphasizes the structures and/or organs of interest. In some instances, it is advantageous to use white or broad spectrum illumination, in conjunction with a multichannel image capturing unit (e.g., RGB sensor). In such cases, the recognition of the structures and/or organs of interest may be improved according to their broad spectrum absorbance profile. Finally, the acquired images may be further analyzed by a machine learning or artificial intelligence algorithm for the system to alter the flow path or the condition of the larvae downstream of the detection region (e.g., separate them according to sex).

Thus, according to one aspect of the invention, there is provided a method for automated detection of structures and/or organs in organisms (such as insects, shrimp or fish) in a flow-system, comprising the steps of: (a) illuminating the organisms in a detection region, (b) imaging the organisms in the detection region, with an extended depth of field that spans at least one-fourth of their thickness, from at least two different directions, thus acquiring optical data; (c) analyzing the optical data for the presence of one or more structures and/or organs of interest.

The feature “extended depth of field” is one of the method advantages and should be understood as follows. An extended depth-of-field imaging system makes it possible to clearly capture a subject without adjusting the distance between the subject and system.

In some embodiments, said detection region is at least a portion of a flow-channel. The term flow-channel may be used intermittently with the term “fluidic channel”.

In other embodiments, the detection region may include an electro-optical module for monitoring the organisms flowing in the at least a portion of the fluidic channel. The electro-optical module comprises at least one sensor (imaging unit).

The channel may be transparent or partially transparent. For example, the flow-channel may be manufactured as a capillary from glass or plastic.

In some embodiments, the organisms of interest (insects, shrimp or fish) flow through said fluidic channel one at a time. In some embodiments, said organisms in said detection region are aligned parallel to said flow-channel.

The inner cross-section of the channel may be selected to be slightly greater than the average thickness of the organism of interest, to allow both the organism's flow through the channel, and its appropriate positioning in the channel while being imaged. For example, the inner cross-section of the channel may be up to of about 120% of the organism's thickness.

In some embodiments, the cross-sectional shape of said flow-channel is square or rectangular. In some embodiments, said flow-channel is positioned between image capturing unit(s) to image said insects, shrimp and fish, and light-source(s) for illuminating them.

The term “image capturing unit” or “imaging unit” may be used intermittently with the term “sensor”.

In some embodiments, the number of said observation directions is two, and the angle between them is 90°. In various embodiments, the number of said observation directions may vary between 2 and 4. When using 2 or 4 directions, an angle of 90 degrees may be recommended between them, to contribute to most representative information. When using 3 directions, then an angle of 120 degrees between them may be recommended.

In some embodiments, the depth of field is at least of about ¼ of the entire thickness of the organism, at each particular observation direction. This parameter is developed due to the fact that the organs of interest may be located close to the surface of the larvae body. In practice, the range of the depth-of-field may be between ¼ and 1 of the organism's thickness, when the organism's thickness is close to the inner size of the flow channel. However, in case the channel is wider than the organism's body thickness, the range of the depth-of-field may be selected accordingly, for example between ½ and 3 or more.

In some embodiments, said at least two observation directions form a plane perpendicular to the flow channel's axis.

In some embodiments, said images (forming the optical data) are pre-processed before being analyzed.

In some embodiments, said images are acquired by the sensors being CCD or CMOS cameras.

In some embodiments, said images taken from different directions are not all simultaneous (for example, when said images are taken at of different locations along the flow-channel).

In some embodiments, said images taken from different directions and/or at different locations are synchronized.

In some embodiments, one said image capturing unit (sensor) may image said organisms from different directions through a system of mirrors.

In some embodiments, said image capturing unit (and/or the whole electro-optical module) is rotatable around the flow-channel.

In some embodiments, said pre-adult insects, shrimp and fish are rotated about the main axis of the flow channel.

In some embodiments, said imaging is performed with an f-number greater than 4.

The depth-of-field may be controlled by changing the f-number and other parameters of the system (for example, optical magnification, camera sensor, etc.) in some embodiments, pulsed (strobed) illumination may be used, thereby reducing heat effect of the illumination and rendering requirements to the camera quality less critical.

The strobed illumination allows using simpler sensors (cameras), e.g. those without a global shutter.

Strobed illumination not only replaces the need for a global shutter, but also allows using powerful pulses which are effective for obtaining high quality images and harmless to the organisms.

Non-strobed illumination of similar power would require a high-power, non-coherent light source in order to prevent interference artifacts such as speckles. This in turn translates into:
A significantly more expensive setup compared to a pulsed LED light source
Requires high-power energy supply
Heating the flow-channel (and the rest of the system) which has a negative effect on the organisms of interest passing through it and on the mechanical integrity of the system.

Based on the above, pulsed illumination is more advantageous, especially when the organisms are heat sensitive. Further, the pulse duration is chosen so as to ensure that the images are not smeared because of the organisms flow through the channel. Specifically, the equation close to the following may be used to calculate the pulse duration:

dT=(N*p*PS)/(v*M) (1)

where dT is the pulse duration, N is the size of the object of interest (in the flow direction), p is the % of allowed smearing for the object of interest (p=n/N where n is the length of the smeared region in pixels in the flow direction), PS is the pixel size of the imaging sensor (e.g., camera), v is the flow velocity of the object of interest in the channel and M is the optical magnification of the imaging system.

The above equation allows determining parameters of the pulsed illumination in order to obtain an image of high quality with a predetermined, modest length of smearing (for example the smearing of about 1-2 pixels only) being invariant to the organism's size.

In other words, the inventors have achieved such a quality of the image, that for example, the size of a larva on the picture may be equal to its real size+1-2 pixels only.

In the proposed technique, the captured images may be smeared by less than 0.25% of the full frame.

In some embodiments, light bursts may be used. These are short pulses having duration of less than 50 microseconds.

In alternative embodiments, relatively low power pulses may be used, if the camera is highly light sensitive. In all cases, the average pixel intensity in the ROI, with no object (organism) present, should be greater than 10% of the saturation value.

The illumination (for example, the pulsed illumination) may be performed by at least one light source which may form part of the mentioned electro-optical module. In some embodiments, the light source duty cycle may be less than 1:100. (where duty cycle D is the relation of duration of a single pulse (pulse width) PW to the total width TW of the signal up to the next pulse: D=PW/TW.)

In some embodiments, said illumination is white or broad spectrum. In some further embodiments, such illumination is the preferred type of illumination.

In addition, short illumination pulses (for example, with a pulse duration of less than 50 microseconds) allow for cost reduction.

In some embodiments, said illumination may be IR or near-IR illumination.

The color of the illumination may be chosen according to each specific application. In some cases, a monochromatic or quasi-monochromatic illumination may be advantageous in emphasizing structures or organs of interest. However, in many instances, there is no essential difference in absorbance or emission between different light colors.

In some cases, the object of interest may have strong absorbance at a certain wavelength or spectrum (for example due to high concentration of molecules which absorb light). It is also possible to encounter the other extreme, low absorbance, which causes the object of interest to appear close to transparent. In some additional cases, the object of interest or its surroundings may be fluorescent. In which case, illuminating them at the correct “excitation” wavelength causes a fluorescent emission at a different color. In case of detecting a strong signal associated with a certain color (for example, in case of self-fluorescence or strong absorbance to a specific light color in sex-organs of a specific organism) illumination at such a specific light color will be preferred.

In many embodiments, white or broad spectrum illumination is selected together with a color camera (e.g., 3 RGB channels), since this combination provides more spectral information regarding the illuminated object (organism) compared to monochromatic or quasi-monochromatic) illumination.

In some embodiments, a plurality of pixel rows of said at least one image capturing unit (sensor) are positioned in parallel to the main axis of said flow-channel.

In some embodiments, only part of the image capturing sensor (actually only some rows of the plurality of pixel rows of said image capturing sensor) is used for acquiring images. Such a solution allows for faster imaging.

In some embodiments, said illumination is collimated.

In some embodiments, said images not containing the organisms of interest are used for adjusting and pre-processing said images containing said organisms.

For example, such images may be used for background subtraction, for reference to determine whether an acquired image contains a larva etc., and for testing and aligning the system.

In some embodiments, a blocking structure (for example, an aperture) is associated with each said image capturing unit, such that only light from said detection region can reach it.

In some embodiments, only a portion of said insects, shrimp or fish is imaged.

In some embodiments, said one or more organs of interest are sex related.

In some embodiments, based on the analysis of said images, the sex of said pre-adult insects, shrimp and fish can be determined.

The presence or absence of a male or female sex organ may be determined by a machine learning (ML) algorithm. The ML algorithm needs high quality pictures. For example, the Inventors may use pictures where the relevant organ in Aedes albopictus L3 larvae is ˜1200 pixels in size. It is still visible at lower resolution (even ˜75 pixels), yet the ML code has a harder time detecting it at the same level of precision.

To determine recommended values of flow velocity, camera magnification etc. for obtaining a high quality of the picture invariant to the organism (e.g., larva) size, the above equation (1) may be used.

In view of the above, the use of pulsed white or broad spectrum illumination from two or more directions, in combination with the imaging having an extended depth of field imaging, allows receiving high quality images suitable even for detecting features/organs of pre-adult organisms, and for further sorting of such organisms.

The sorting may be focused, for example, on separation of strong organisms from weak ones, alive ones from dead ones, fast developing ones from retarded ones etc.

Alternatively or in addition, the sorting may include sex separation of the organisms of interest.

In some embodiments, based on the analysis of said images and downstream of said detection region, a controller coupled to the flow-system can alter the flow path or the condition of the organisms such as shrimp, fish or pre-adult insects.

For example, as the pre-adult organisms (say, larvae) flow through the system one by one, each one can be directed to the appropriate outlet (e.g., males only, females only, uncertain). This may be done using valves which control the flow path.

In addition, the technique may comprise choosing to alter the condition of certain larvae (e.g., to destroy them, sterilize them by irradiation) by using the same flow system, and after the sex has been determined.

All features of the technique, described above, define both the method and a suitable system (see below), mutatis-mutandis.

Based on the above-mentioned broad aspects of the invention, there are provided more specific methods and systems, for example:

A computer-implemented method for automated detection of features, structures and/or organs in organisms being shrimp, fish or pre-adult insects, comprising steps of:
(a) ensuring flowing of said organisms via a detection region of a flow system,
(b) illuminating the organisms in the detection region,
(c) imaging an individual organism in the detection region by one or more sensors, with an extended depth of field that spans at least one-fourth of the organism's thickness, from at least two different observation directions, thereby obtaining optical data in the form of images;
(d) analyzing the obtained images for the presence of one or more said structures and/or organs of interest in said organism.

A suitable flow-system for automated detection of said features, structures and/or organs in organisms being shrimp, fish or pre-adult insects, the system comprising:
(a) a fluidic channel configured to allow flowing there-through of the organisms suspended in liquid media, the fluidic channel including a detection region,
(b) an electro-optical module for monitoring the organisms in the detection region, including:
   an illumination unit configured for illuminating the organisms in the detection region,
   an imaging unit comprising one or more sensors configured for acquiring optical data by capturing images of an individual organism in the detection region from at least two different observation directions and with an extended depth of field that spans at least one-fourth of the organism's thickness,
(c) a processor in communication with the electro-optical module, configured for receiving the optical data from said module, processing and analyzing thereof for the presence of one or more said structures and/or organs of interest in the organism.

Further, there is a computer implemented method of sorting organisms being shrimp, fish or pre-adult insects, comprising:
a) providing a flow system comprising a fluidic channel with an inlet, a detection region and at least one outlet, an electro-optical unit for monitoring the detection region, a processor and a controller;
b) ensuring passage of the organisms suspended in liquid media through the inlet into the fluidic channel towards the detection region;
c) acquiring optical data of the individual organism by imaging thereof at the detection region by one or more sensors of the electro-optical module;
d) transmitting the optical data of the individual organism to the processor;
e) processing the optical data and performing classification of the individual organism based on one or more morphologic features and/or color-related features and/or sex related features (for example, a set of parameters indicative of the sex), extracted from the optical data;
f) providing instructions to the controller based on the classification of the individual organism, and
g) sorting the organism according to the instructions received by the controller.

It should be noted that the sorting of the organisms based on the morphologic or color-related features (such as color, length, thickness, height etc.), may be used in order to select strong, and healthy individual organisms. In addition or instead of the above, some specific, sex-related features/parameters may be used for sex separation of the organisms.

There is also provided a suitable flow-system for sorting of organisms being shrimp, fish or pre-adult insects, the system comprising:
a fluidic channel comprising an inlet, a detection region and at least one outlet, said channel is configured to allow flowing there-through of the organisms suspended in liquid media;
a processor;
an electro-optical module for monitoring the detection region, being in communication with the processor,
a controller in communication with said processor;
   wherein
the electro-optical module comprises at least one sensor configured to acquire optical data of an individual organism by imaging thereof, and to transmit said optical data to the processor,
the processor is configured
   to receive and process the acquired optical data,
   to perform classification of said organism based on one or more morphologic features and/or sex-related features (for example, a set of parameters indicative of the sex), and
   to instruct the controller to sort said organism based on the classification.

In other main aspects, the present invention provides a computer implemented algorithm comprising code to perform the steps of the method as defined in any of the above-described methods.

Computer readable non-transitory storage medium is also provided, storing computer-implementable instructions of said code.

It is to be acknowledged that the text on pages 3 to 58 of the Inventors' WO2021/038561 is incorporated herein by reference.

The present invention is a novel, rapid and cost effective technique of analyzing and possible sex-sorting of the mentioned organisms, such as pre-adult insects and shrimp, and fish in any stage (whether in the larva or fry or fingerling or adult developmental stages). The process as inter-alia disclosed is automated and preferably does not require user intervention.

The terminology used in the present description and claims.

The term “organisms” as used herein refers to fish, shrimp and insects. The insects of interest are mainly in the pre-adult shape, to allow their survival in the proposed flow system. The shrimp should be understood as a decapoda group comprising various shrimp, prawns and crawfish.

The term “fish” as used herein generally refers to gill-bearing aquatic craniate animals.

The term “fluidic channel” as used herein refers to a device, apparatus or a container having an inner space and outer walls, wherein the inner space is contained with and/or allow the passage of a flowable fluid or liquid media or solution, for example water or isotonic aqueous solution. Such a fluidic channel has at least one inlet and at least one outlet and in the context of the present invention is adapted to contain organisms of interest, suspended in a liquid media passing or flowing from the inlet portion of the channel towards the outlet portion of the channel while subjected to automatic/computed analyzing and/or classification process.

The terms “movement”, “flowing” or “passage” in the context of the invention is meant to be understood as voluntary or un-voluntary movement of the organism suspended in liquid media, as it passes through the channel.

The term “streaming” herein refers to flowing or rapidly flowing of a liquid or fluid or suspension, e.g., a velocity or volume flow in a channel. The term “streaming” covers both the case of passive flow, and the case of active flow of liquid media in the fluidic channel.

The term “liquid media” refers, without limitation, to a substance in a physical state in which it does not resist change of shape but does resist change of size. The term “passive flow” refers to flow control methods which require no auxiliary power. The non-limiting list of passive flow control methods includes any passive devices which are steady and require no energy by definition (e.g., derived by gravity forces), such as turbulators, roughness elements or any other passive device influencing the fluid dynamics of the flow.

The term “active flow” refers to flow control methods which require energy expenditure. The non-limiting list of active flow control methods includes valves, pumps, pressurizing means, applying a positive or negative (vacuum) pressure difference with respect to parts of the system of the present invention or any other artificially driven actuator which require energy.

It is within the scope of the present invention that the system and method for sex-separation or sorting of organisms comprises means to drive them out of a container containing unsorted organisms and through the system that may be connected to such a container towards the outlet of such a system. These include vacuum, valves, gravity forces and pumps. These means may further include a controllable guiding tool configured to guide the organisms to an outlet associated with the acquired classification. Examples of such a guiding tool may comprise one or more valves, a transducer configured to generate acoustic radiation forces, electrodes configured to apply electroosmotic forces to the liquid media, electrodes configured to apply dielectrophoretic forces to the organism and any combination thereof.

The term “controller” refers, without limitation, to any hardware device or a software program, or a combination of the two that manages or directs the flow of data between two or more entities. In a general sense, a controller can be thought of as something or someone that interfaces between two systems and manages communications between them. The system of the present invention may comprise a control center, wherein said control center is configured to monitor the system performance.

It is within the scope of the present invention that the system, e.g., using the detection/classification region, processor and controller parts is configured to classify an individual organism into classes selected from a non-exhaustive list comprising male, female, and/or other by a set of parameters indicative of the sex of the organism (e.g., based on optical and/or other data of the organism).

Alternatively or in addition, the above-mentioned list may comprise other classes, defined by other attributes (e.g., dead or alive; healthy or unhealthy, for example based on color and color transmission) or by probability of future attributes (e.g., a class of estimated mature size, for example based on previous statistics data and the current size).

Terms “features”, “parameters”, “attributes” are intermittently used in the description as forming criteria for analyzing and/or sorting (classification) of the organisms. As used herein, the features/parameters/attributes for the analyzing and/or sorting (classification) include one or more features from the following three groups defined in a non-limiting manner:
a first non-exhaustive group of morphologic features/parameters/attributes, comprising: overall size of the organism, morphology of the organism, shape, segment size ratio,
a second non-exhaustive group of color-related features comprising: absorption, transmission, IR (Infrared) absorption, LR transmission, color, fluorescence,
a third non-exhaustive group of mainly sex-related features/parameters/attributes, comprising: gonad disc morphology, secondary sex organ morphology, gonad size, gonad morphology, gonad autofluorescence, size of testes, size of male accessory glands, morphology of testes, morphology of male accessory glands, autofluorescence of testes, autofluorescence of male accessory glands, size of the developing male and female primary or secondary reproductive structures, primitive sex organs, morphology of the developing male and female primary or secondary reproductive structures, autofluorescence of the developing male and female primary or secondary reproductive structures and any combination thereof.

Applicability of each of the groups to different types of sorting will be discussed later.

The term “Stroboscopic illumination system” refers to a system capable of emitting short pulses of light (Imicrosecond-10 milliseconds). The pulses of lights can be generated by: light emitting diodes (LEDs), lasers, solid state light sources and Xenon lamps. This system minimizes illumination time, thus reducing generated heat, phototoxicity and photobleaching artifacts as well as blurring due to motion.

The term “High Frame Rate Camera” refers to a camera capable of capturing images with exposures of less than 1/1,000 second or frame rates in excess of 30 frames per second. It might be useful for recording fast-moving objects as photographic images onto a storage medium.

The term “system performance parameters” refers to key system capabilities that must be met in order for a system to meet its operational goals. A non-limiting list of system performance parameters of the invention includes: flow rate, sorting rate, sorting accuracy, organism count, specificity, sensitivity, survival, fitness and throughput.

The terms “accuracy”, “specificity” and “sensitivity” of a test may be defined by in terms of TP, TN, FN and FP as detailed below. In the context of the present invention, the tested parameter may be male or female organism.

True positive (TP)=the number of cases correctly identified as positive for the tested parameter.

False positive (FP)=the number of cases incorrectly identified as positive for the tested parameter.

True negative (TN)=the number of cases correctly identified as negative for the tested parameter.

False negative (FN)=the number of cases incorrectly identified as negative for the tested parameter.

“Accuracy”: The accuracy of a test is its ability to differentiate the positive and negative cases correctly. To estimate the accuracy of a test, the proportion of true positive and true negative in all evaluated cases should be calculated. Mathematically, this can be stated as:

Accuracy=(TN+TP)/(TN+TP+FN+FP)=(Number of correct assessments)/Number of all assessments)

Sensitivity: The sensitivity of a test is its ability to determine the cases positive for the tested parameter correctly. To estimate it, the proportion of true positive in cases evaluated positive for the tested parameter should be calculated. Mathematically, this can be stated as:

Sensitivity=TP/(TP+FN)=(Number of true positive assessment)/(Number of all positive assessment)

Specificity: The specificity of a test is its ability to determine the cases negative for the tested parameter correctly. To estimate it, the proportion of true negative in cases evaluated negative for the tested parameter should be calculated. Mathematically, this can be stated as:

Specificity=TN/(TN+FP)=(Number of true negative assessment)/(Number of all negative assessment)

It is herein acknowledged that morphology is a branch of biology dealing with the study of the form and structure of organisms and their specific structural features. This includes aspects of the outward appearance (shape, structure, color, pattern, size), i.e. external morphology (or eidonomy), as well as the form and structure of the internal parts like bones and organs, i.e. internal morphology (or anatomy). Morphology is a branch of life science dealing with the study of gross structure of an organism or taxon and its component parts.

The term “electro-optical module” as used herein refers to a module or unit or region comprising devices (e.g., lasers, LEDs, waveguides etc.) and/or systems and/or sensors which operate by the propagation and interaction of light (electromagnetic or optical) and the electrical (electronic) state. Electro-optical sensor is an electronic detectors that convert light, or a change in light, into an electronic signal. These sensors are able to detect electromagnetic radiation from the infrared up to the ultraviolet wavelengths. An optical sensor converts light rays into electronic signals. Without wishing to be bound by theory, the electro-optic effect is a change in the optical properties due to interaction with light. Such a module may serve the functions of light modulation, signal routing, tunable filtering, and polarization conversion. They have potential applications such as wide-band optical communication, optical signal processing, and sensors. In the context of the present invention, the electro-optical module of the detection region comprises at least one of: one or more light sources configured to illuminate the classification region, one or more acoustic sources configured to generate sound waves that pass through the classification region, one or more image sensors, at least one optical element and an internal control unit in-communication with the processor.

According to one embodiment, the electro-optical module comprises more than one light source. In certain aspects, each light source is configured to emit light with predetermined spectrum and/or intensities. More particularly, illumination may be broadband (for example, white light) or monochromatic.

The term “sensor” as used herein generally refers to a device, module, machine, or system capable of detecting or measuring a property or changes in its environment and sending the information or data (e.g., optical or image or acoustic data) to other electronics, frequently a computer processor. Non limiting examples of sensor types within the scope of the present invention include, but are not limited to acoustic, sound and/or vibration sensors, electric current, electric potential, magnetic and/or radio sensors, flow and/or fluid velocity sensors, optical, light, imaging and/or photon sensors, pressure sensors, thermal, heat and/or temperature sensors and position/location sensors.

The term “optical sensor” or “optical element” as used herein is meant to include photoconductive devices which convert a change of incident light into a change of resistance, photodiodes which convert an amount of incident light into an output current, phototransistors are a type of bipolar transistor where the base-collector junction is exposed to light. Optical sensors within the scope of the present invention may include: optical detector, a camera, a photodiode, a photomultiplier, an image acquisition sensor, an optical acquisition sensor, an electro-optical sensor, light detector, a photon sensor, a reflectometer, a photodetector, a spectral image sensor, and any combination thereof.

This term further encompasses an optical element such as a lens, a mirror, a polarizer, an excitation filter, an emission filter, a dichroic mirror, an optical coating such as antireflective coating, an optical grating, at least one stereoscopic microscope, at least one fluorescence microscope and any combination thereof.

The term “imaging sensor” or “image sensor” or “image acquisition sensor” as used herein refers to a sensor that detects and conveys information used to make an image. Without wishing to be bound by theory, an imaging sensor conveys the variable attenuation of light waves, passed through or reflected off objects, into signals, that convey the information. The waves can be light or other electromagnetic radiation. Image sensors are used in electronic imaging devices of both analog and digital types, which include digital cameras, camera modules, camera phones, and others.

Exemplary imaging sensors within the scope of the present invention include: RGB (red, green, blue) frequency spectrum, broad spectrum, hyperspectral, visible light frequency range, near infrared (NIR) frequency range, infrared (IR) frequency range, monochrome, specific light wavelengths (e.g., LED or laser and/or laser and/or halogen and/or xenon and/or fluorescent), UV frequency range, a reflectometer and combinations of the aforementioned.

As used herein, the term “optical data” generally refers to data retrieved from optical media which can be stored on an optically readable medium. Examples of optical storage devices, include CD, DVD and Blu-ray discs etc. In other aspects it refers to an optically formed reproduction of an object, such as one formed by a lens or mirror. The term optical data within the context of the present invention encompasses image data.

The term “image data” herein means a photographic or trace objects that represent the underlying pixel data of an area of an image element, which is created, collected and stored using image constructor devices. Image data attributes include for example, image resolution, data-point size and spectral bands. In the context of the present invention the term “computer” stands for but no limited to a machine or device that performs processes, calculations and operations based on instructions provided by a software or hardware program.

The term “computer” also means in the context of the present invention a control unit or controller. It is designed to process and execute applications and provides a variety of solutions by combining integrated hardware and software components. The computer of the invention is configured to extract a predetermined set of feature vectors from the image data of the organism; to compute sex-characteristics of the organism based on the set of feature vectors, attributes or parameters; to generate sex-classification output and to transmit the output to the controller unit.

The present invention may be a system, a method, and/or a computer program product. The computer program product may include a computer readable storage medium (or media) having computer readable program code/instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing.

A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

According to certain aspects, the method of the present invention comprises steps of applying a machine learning process with the computer implemented trained algorithm to detect organs and/or to determine the sex of an organism. Thus, it is within the scope of the present invention that the algorithm (or computer readable program) is implemented with a machine learning process using a neural network with the processed data.

The term training in the context of machine learning implemented within the system of the present invention refers to the process of creating a machine learning algorithm. A source of training data can be used to train machine learning models for a variety of use cases, from failure detection to consumer intelligence.

The neural network may compute a classification category, and/or the embedding, and/or perform clustering, for identifying sex of an individual organism i.e. male or female.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks herein disclosed.

As used herein, the term “classifying” may sometimes be interchanged with the term clustering or tagging, for example, when multiple organism's images are analyzed, each image may be classified according to its predefined features or feature vectors and used to creating clusters, and/or the images may be embedded and the embeddings may be clustered.

The term “classification” category may sometimes be interchanged with the term embedding, for example, the output of the trained neural network in response to an image of an organism may be one or more classification categories, or a vector storing a computed embedding. It is noted that the classification category and the embedding may be outputted by the same trained neural network, for example, the classification category is outputted by the last layer of the neural network, and the embedding is outputted by a hidden embedding layer of the neural network. The architecture of the neural network(s) may be implemented, for example, as convolutional, pooling, nonlinearity, locally-connected, fully-connected layers, and/or combinations of the aforementioned.

It is noted that the tagging and classifying of the organism in the images or the sex-sorting characteristic targets may be manually or semi manually entered by a user (e.g., via a graphical user interface, for example, selected from a list of available phenotypic characteristic targets), obtained as predefined values stored in a data storage device, and/or automatically computed.

The term “feature vector” refers hereinafter in the context of machine learning to an individual measurable property or characteristic or parameter or attribute of a phenomenon being observed e.g., detected by a sensor. It is herein apparent that choosing an informative, discriminating and independent feature is a crucial step for effective algorithms in pattern recognition, machine learning, classification and regression.

Algorithms using classification from a feature vector include nearest neighbor classification, neural networks, and statistical techniques.

In computer vision and image processing, a feature is an information which is relevant for solving the computational task related to a certain application. Features may be specific structures in the image such as points, edges or objects. Features may also be the result of a general neighborhood operation or feature detection applied to the image.

When features are defined in terms of local neighborhood operations applied to an image, a procedure commonly referred to as feature extraction is executed.

Although embodiments of the disclosure are not limited in this regard, discussions utilizing terms such as, for example, “processing”, “computing”, “communicating”, “training”, “capturing”, “executing”, “calculating”, “feeding”, “determining”, “establishing”, “analyzing”, “checking”, “tagging”, “classifying”, “transmitting”, “exerting” or the like, may refer to operation(s) and/or process(es) of a computer, a computing platform, a computing system, a computer implemented algorithm or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes.

According to one embodiment, the present invention provides a device, system and method for sex-sorting of pre-adult insects as well as of shrimp and fish in any stage (whether in the larva or fry or fingerling or adult developmental stages). In some of its aspects, the system comprises a fluidic channel comprising an inlet and a number of outlets; a classification region and a separation or destruction region. The channel is configured to receive organisms suspended in a liquid. The organisms pass through a detection (classification) region where they are imaged and classified according to their sex by a processing unit. According to this classification, in some embodiments, the organisms are separated into different outlets, where they can then be collected. In some embodiments, the organisms are classified according to their sex with one class passing undamaged to the single outlet for collection, and the rest being destroyed.

It is within the scope of the present invention that a liquid, typically water, containing an organism enters the system, through the inlet and flows, under pressure or gravity, towards the outlet(s). The inner dimensions of the flow channel may be designed such that only a single organism passes at a time. Furthermore, these dimensions may be set so as to limit motion of the organism (e.g., a narrow enough channel constricts it to extend parallel to the channel walls). The temperature of the liquid may be changed or controlled, namely heated (elevated) or cooled (reduced) according to the need. One such use may be to control or affect movements of the organism during analyzing, sorting or separating process (i.e. cooled to reduce wriggling movements and heated to increase wriggling movements). In one embodiment, the temperature of the liquid may be cooled so as to reduce wriggling movements. As the organisms pass through the classification region, they are imaged. The pictures/images are analyzed by a processing unit and the organism may be then classified.

In some embodiments, a separation region is used where each individual is guided to the outlet associated with its class. This may be done by leaving only the appropriate outlet open and closing the rest. Alternatively, all outlets may be left open and one or more of the following techniques may be used to guide each individual to the appropriate one: (1) acoustic radiation forces generated by an external transducer; (2) electroosmotic forces generated by electrodes (forces applied on the liquid): (3) dielectrophoretic forces generated by electrodes (forces applied on the organisms) or (4) a controlled liquid flow from one more additional directions toward a selected outlet of the fluidic channel. Such flows may be created at a so-called separation region where the channel is divided into two or more outlets.

The outlets themselves may also be designed to have different hydrodynamic resistance.

Thus, when no external force is applied, the organisms flow to the outlet with the lowest resistance.

In some embodiments, the number of individuals dispensed into each outlet channel(s) is monitored. This is particularly important when one channel is connected at its end to a container to be used for release.

In some embodiments, a destruction region, through which all individuals pass, is used. The organisms identified as belonging to the class that is needed for the application (e.g., males or females, according to the purpose/application), pass unharmed. The rest (also herein referred to as ‘other’ or ‘unspecified’ or ‘unclassified’) are destroyed. This may be done by at least one of the following means: a laser, a high-power electric field, ultrasonic blasts or by mechanically grinding or squashing the organism. In some embodiments, the number of individuals dispensed unharmed is monitored.

According to further embodiments of the present invention, an electro-optical setup (module) next to the detection region may be used to recognize the sex of the organism, for example fish. It includes at least one of: one or more light sources to illuminate the detection region (possibly from different directions and in different colors); one or more image sensors, to photograph the detection region (possibly from different directions); an optical setup, comprising one or more optical elements, e.g., lenses, mirrors, polarizers, excitation filters, emission filters, dichroic mirrors, optical coatings (e.g., antireflective coatings) and optical gratings, located between the light source(s) and the detection region and/or the classification region and the image sensor(s); and a connection to a processing unit. In some embodiments, the optical setup is comprised of one or more stereoscopic microscopes and/or fluorescence microscopes and the image sensors are cameras attached to the microscopes.

The organisms may be classified at least into two classes, wherein said two classes being selected from at least the following pairs: male and non-male, female and non-female, male and female, alive and dead, healthy and sick, strong and weak, quickly developing or retarded. In the sex classification, three classes may exist (male, female and undetermined).

According to some embodiments, pictures taken by the image sensors are analyzed in the processing unit to determine the class of an individual organism. One or more visual attributes/parameters/features from at least one of the following first second and third non-exhaustive groups may be used for analyzing the organisms and also for sorting the organisms according to a desired classification.

For example the first and second groups of morphologic and color-related features may be applied, comprising: overall size of the organism, morphology of the organism, shape, segment size ratio, absorption, transmission, IR (Infrared) absorption, IR transmission, color, fluorescence.

These two groups of morphologic and color-related features/attributes/parameters may be applied to any type of sorting (i.e. alive-not alive, healthy-sick, strong-weak, male-female etc.).

Alternatively or in addition, the third group of sex-related features/attributes/parameters may be applied, comprising: gonad disc morphology, secondary sex organ morphology, gonad size, gonad morphology, gonad autofluorescence, size of testes, size of male accessory glands, morphology of testes, morphology of male accessory glands, autofluorescence of testes, autofluorescence of male accessory glands, size of the developing male and female primary or secondary reproductive structures, primitive sex organs, morphology of the developing male and female primary or secondary reproductive structures, autofluorescence of the developing male and female primary or secondary reproductive structures and any combination thereof.

The third group of attributes/features/parameters may be applied to the sex-sorting.

In other words, it is within the scope of the present invention that differences between male and female organisms may be sorted by one or more attributes (parameters indicative of the sex) including, but are not limited to, the overall size of the organism, morphology of the organism, shape, segment size ratio, absorption, transmission, IR (Infrared) absorption, IR transmission, color, fluorescence, gonad disc morphology, secondary sex organ morphology, gonad size, gonad morphology, gonad autofluorescence, size of testes, size of male accessory glands, morphology of testes, morphology of male accessory glands, autofluorescence of testes, autofluorescence of male accessory glands, size of the developing male and female primary or secondary reproductive structures, primitive sex organs, morphology of the developing male and female primary or secondary reproductive structures, autofluorescence of the developing male and female primary or secondary reproductive structures and any combination thereof.

According to further aspects of the present invention, classification may be carried out automatically using one or more of the following techniques: image processing, machine learning and trained neural network. The neural network may be a classical neural network or a deep network (deep learning network), including a convolutional neural network, or any other kind of neural network.

In other aspects of the present invention, the flow velocity of each organism may be monitored in order to determine when they reach the separation region and need to be guided to the appropriate outlet or reach the destruction region and need to pass freely or be destroyed.

In some embodiments, this is done by taking successive images in the detection (classification) region. In some embodiments, a flow-meter or an additional camera with a wide field of view, are used for tracking the organisms along the system. Reference is now made to figures in which exemplary non-limiting embodiments are described.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 illustrates one example of the proposed flow-system for determining structures or organs of interest and/or for classifying organisms such as pre-adult insects, shrimp or fish.

FIGS. 2A-2C schematically illustrate non-limiting examples of a flow system with integrated detection/classification region and sorting region (FIGS. 2A-B) and optionally destruction region (FIG. 2C), according to some embodiments of the present invention;

FIG. 3 is an illustrative flow chart of one version of the proposed technology, describing an exemplary method and an exemplary flow system for classifying a specific type of the organisms of interest and sorting them accordingly;

FIG. 4 illustrates a non-limiting example of an imaging unit of an electro-optical module according to the present invention;

FIGS. 5A-5B schematically illustrate two exemplary embodiments of the imaging units arranged along the flow system;

FIG. 6 schematically illustrates an exemplary embodiment of the optical arrangement in the flow system, where a single camera can image more than one direction with the use of mirrors:

FIG. 7 schematically illustrates an exemplary embodiment of the optical arrangement in the flow system, where a camera can image more than one direction by rotating it about the detection region.

FIG. 8 schematically illustrates the proposed way of imaging, where the depth of field is at least ¼ of the thickness of the organism of interest.

### DETAILED DESCRIPTION OF SPECIFIC EMBODIMENTS

Embodiments of the proposed technique will be illustrated and commented by referring to a non-limiting example of detecting sex organs in pre-adult organisms, and on non-limiting example of further sex separation of the organisms.

The invention is applicable to other embodiments or of being practiced or carried out in various ways. Also, it is to be understood that the phraseology and terminology employed herein is for the purpose of description and should not be regarded as limiting.

The invention provides, in some of its aspects, a novel fluidic channel comprising an inlet and outlet(s); a detection/classification region or unit or zone or module. Optionally, it also provides a separation region or unit or zone or module and/or a destruction region or unit or zone or module. The channel is configured to receive pre-adult insects or shrimp or fish in any stage (whether in the larva or fry or fingerling or adult developmental stages). suspended in a liquid. In more specific examples, the pre-adult insects may be in the shape of a larva or a pupa or a nymph; the shrimp may be in the shape of a larva or a post-larva or a juvenile; and the fish may be in the shape of a larva or a fry or a fingerling or an adult.

The organisms, pass through the detection/classification region where they are imaged, analyzed and optionally classified according to their sex. According to this classification, in some embodiments, the organisms are separated into different outlets.

FIG. 1 shows a schematic block diagram of one embodiment of the present invention, intended for analyzing organisms like pre-adult insects, shrimp and fish in a flow system 10. The figure shows a fluidic channel 24 through which individual organisms 26 flow from left to right. Their flow may be passive or active. The channel 24, or part of it, may be manufactured of glass. (The organisms may be introduced in the fluidic channel from a container 22 which is shown in dashed lines). When the organisms pass through the detection region 28 of the channel, they are monitored. The monitoring may be provided by an electro-optical module which is schematically marked with the same number 28. For monitoring, the organisms in the channel 24 are usually illuminated. The electro-optical module then performs imaging of an individual organism using one or more optical sensors, and transmits the obtained optical data to a processor 32. The processor analyzes the received optical data (the images) and detects, for example, presence or absence of structures and/or organs of interest on the imaged organism. Additional steps may be taken according to results of the detection process (for example, steps of sex classification and separation) while the imaged organism continues flowing though the channel. Such steps will be illustrated and described in the following figures.

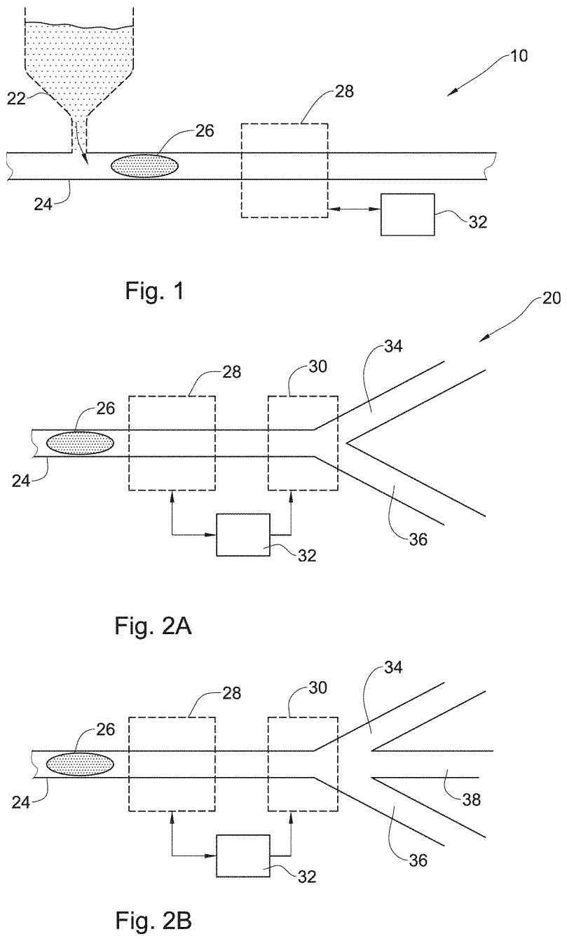

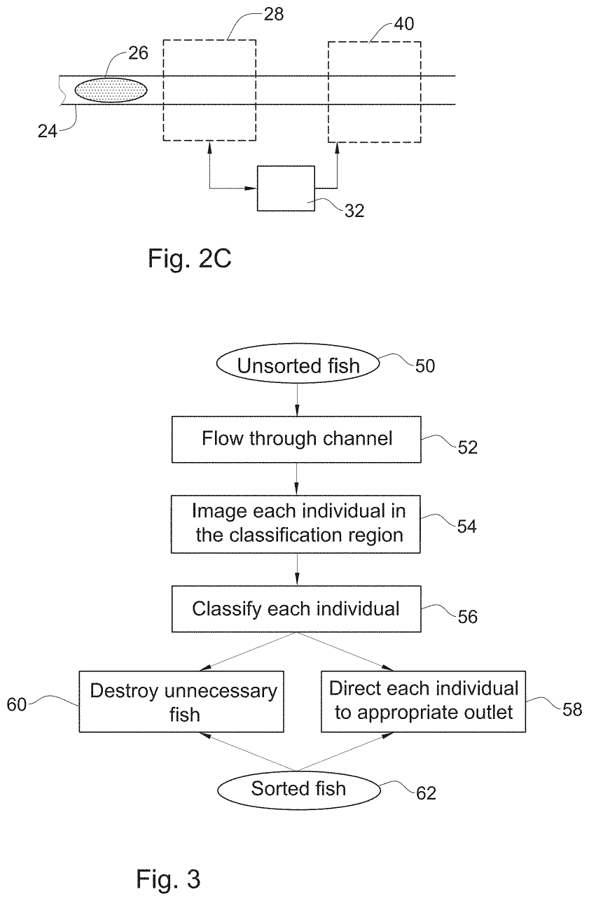

FIGS. 2A-C show schematic illustrations of exemplary flow-systems for sorting organisms like pre-adult insects, shrimp and fish according to the present invention. Upon the imaging of individual organisms within the detection region 28 (which may also be called a classification region), the processing unit/processor 32 performs analysis and classification thereof while the organisms continue flowing until reaching a separation region 30. In the separation region 30, the individual organisms are guided to different channels (for example, by a number of controlled flows) according to the classification performed by the processor 32. The processor 32 may be in control communication with a controller and/or suitable devices (not shown) of the flow system. Classification attributes exist for any organism of interest. For example, such attributes may include, but are not limited to the overall size of the organism, morphology of the organism, shape, segment size ratio, absorption, transmission, IR (Infrared) absorption, IR transmission, color, fluorescence, gonad disc morphology, secondary sex organ morphology, gonad size, gonad morphology, gonad autofluorescence, size of testes, size of male accessory glands, morphology of testes, morphology of male accessory glands, autofluorescence of testes, autofluorescence of male accessory glands, size of the developing male and female primary or secondary reproductive structures, primitive sex organs, morphology of the developing male and female primary or secondary reproductive structures, autofluorescence of the developing male and female primary or secondary reproductive structures and any combination thereof.

FIG. 2A schematically illustrates a flow system 20, where the individual organisms are guided to either a ‘male-only’ or ‘female-only’ channel 34 or an ‘anything else’ channel 36. FIG. 2B shows a schematic illustration, where the individual organisms may be guided to either a ‘male-only’ channel 34, a ‘female-only’ channel 38 or to an ‘anything else’ channel 36. FIG. 2C shows a schematic illustration, where after the classification region only a single class of organisms continue flowing, undamaged, through the main channel. The rest are destroyed in a destruction region 40.

FIG. 3 illustrates a flow chart schematically showing one version of the process according to the present invention. A method for male-female sorting in a flow system may begin with the provision of an unsorted organism (for example, fish) as shown in step 50.

Within the system, the individual organism flows preferably in a single-file towards the detection region (which may also serve as a classification region), as shown in step 52.

There, each individual fish is imaged for detection and/or classification purposes as shown in step 54 of the flow chart.

In step 56 each individual is classified. Classes used may include males and the rest (other, unspecified, unclassified or anything else), or males, females and the rest (other, unspecified, unclassified or anything else) or females and the rest (other, unspecified, unclassified or anything else). Next, the individual organisms reach a separation region, where they are guided, according to their classification, to a predefined flow outlet (step 58). Alternatively, if only a single class is needed, the unnecessary individuals may be destroyed in the channel (step 60). The outcome of the above described process is sex-sorted organisms (step 62).

Further in the present invention, a compact, cost effective, rapid and easy to use technique for detection of structures and/or organs in pre-adult insects, shrimp and fish, is described. Preferably, the organisms of interest are transparent or partially transparent (like larvae or the like), though they may be non-transparent (like some adult fish).

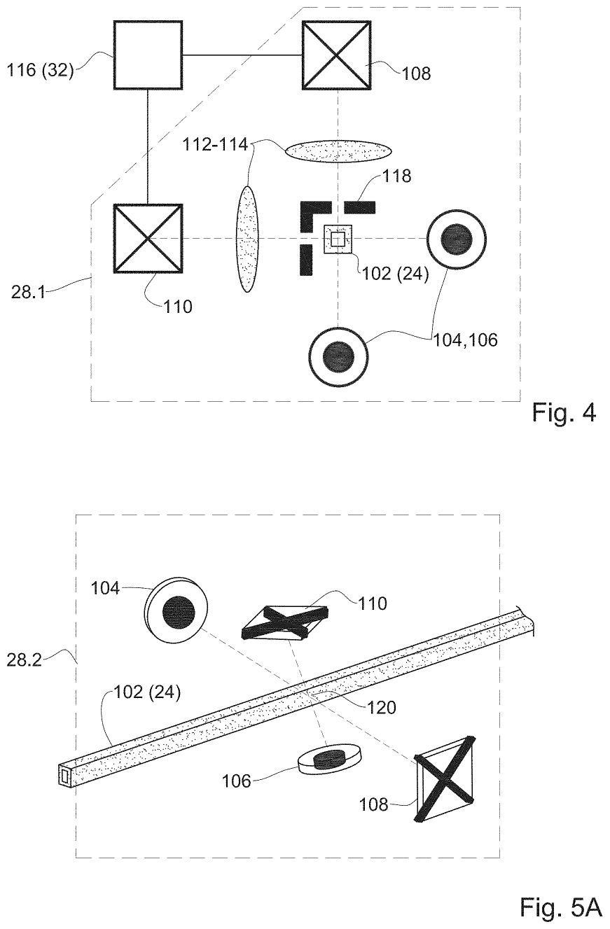

A first exemplary embodiment of automated detection of structures in the mentioned organisms (say, pre-adult) in a flow channel by imaging is shown in FIG. 4.

FIG. 4 is a schematic cross-section of the flow system at its detection region. The detection region includes a portion of the fluidic channel 102 (24) surrounded by components of an electro-optical module schematically marked 28.1. The organisms, for example in the form of larvae, flow one at a time through a transparent square capillary (102, 24), where they are illuminated, from two different directions, by light sources (104, 106) [all shown from top-view]. Images of the organisms are captured by image capturing units (108, 110), such as CCD or CMOS sensors, after being focused by a single lens or an array of lenses (112, 114). When two image capturing units are used, the angle between their observation directions is preferably 90° as this is the angle with the greatest information gain. In another particularly preferred embodiment of the present invention, observation takes place from three or more directions. The acquired images are transmitted to a processing unit (116, 32) where they may be preprocessed before being analyzed by an image recognition algorithm. The processing unit 116 may also be in communication with the flow-system control module (30, see FIG. 2A). As mentioned, it can alter, based on the algorithm's results, the flow path or the condition of the organisms downstream of the detection region (e.g., separate the larvae into different containers according to their sex, destroy some classified organisms, sterilize some classified organisms by irradiation, etc.)—The color of the illumination is chosen according to the level of penetration in the organism analyzed and the level of highlighting of the structures and/or organs of interest. In some instances, it is advantageous to use white or broad spectrum illumination, in conjunction with a multichannel image capturing unit (e.g., RGB sensor) which provides more information than a grey-scale sensor. In such cases, the recognition of the structures and/or organs of interest may be improved according to their broad spectrum absorbance profile. In other instances, using Infra-red (LR) or near-IR illumination, may be beneficial when visible light is strongly attenuated as it passes through the organism (for example when the organisms are non-transparent or only partially transparent). In such instance, the IR or near-IR illumination could be used as the sole illumination source(s) or alongside visible illumination as well.

In this embodiment, a blocking structure (118), for example an aperture associated with the image capturing units ensures that only light (depicted by dashed line) from the flow-channel can reach them. Furthermore, the image capturing units and light sources are placed one opposite the other, with the capillary in between.

Since the image capturing units 108, 110 (cameras, sensors) are imaging the moving (flowing) larvae, steps should be taken to make sure that the resulting images are sharp and not smeared. One approach would be to use a camera with a fast, global shutter (<50 uSec) and a light source constantly turned on. The problem with this approach is that as the camera sensor is illuminated for only a short time during each frame acquisition, either the light source needs to be powerful or the camera sensor extremely light-sensitive. Both these options are expensive and generate additional problems. Light-sensitive sensors typically have lower resolution (less pixels) while powerful light sources generate a lot of heat. The Inventors' solution is to use pulsed (strobed) illumination. The outcome is the same as in the previous case—the camera sensor is illuminated for only a short time during each frame acquisition—yet much simpler and cheaper to implement. First, one could use a common LED light source to generate light bursts (<50 uSec). Second, so long as the light source duty cycle is small (for example, it may be less than 1:100), there is very little heat generation. Finally, there is no special requirement on the camera in the system (e.g., light sensitivity, speed of shutter (could be in the millisecond range) and type of shutter (a simple rolling shutter would work fine)).

An RGB sensor/camera can be used.

The illumination pulses may be synchronized to take place in the periods when the image sensor performs the imaging (and not when the image sensor saves the pictures to memory).

Smearing of the obtained images may be minimized down to 1-2 pixels by selecting a suitable duration of the illumination pulses, for example using an equation (1). In percent, the smearing of the obtained images may be less than 0.25% of the full frame. The illumination pulse duration may be less than 50 microseconds.

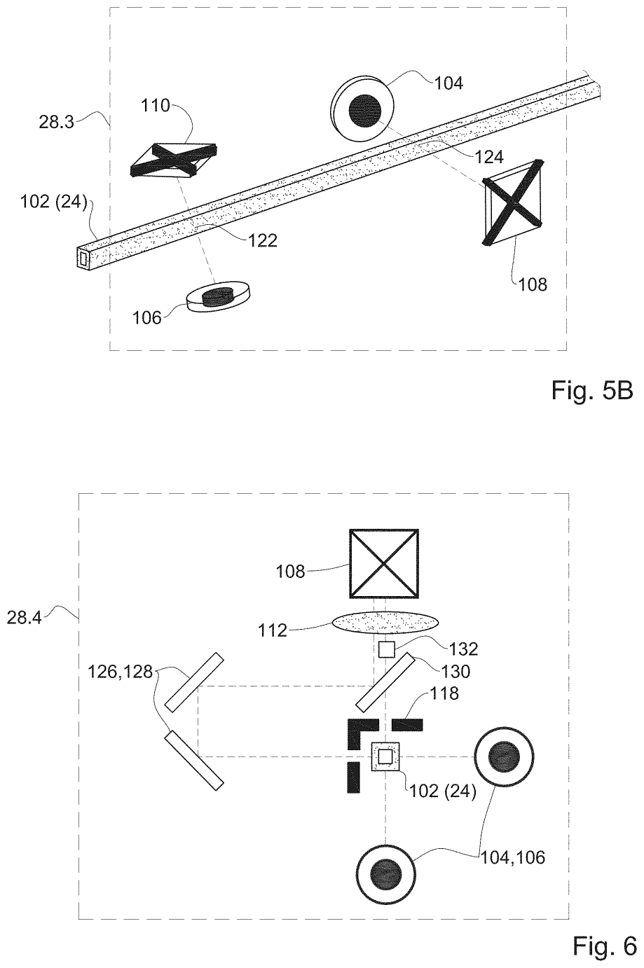

FIG. 5 shows two different embodiments for the automated detection of structures in the organisms of interest in a flow-channel [both in isometric-view]. The detection is performed using measurements of the electro-optical module indicated 28.2. In FIG. 5A, the different image capturing units (two are shown) image the organism at the same location along the capillary (120). In FIG. 5B, the different image capturing units (two are shown), belonging to the electro-optical unit 28/3, image the organism at different locations along the capillary (122, 124).

The images of one and the same object taken from different locations along the flow channel may be not all simultaneous.

If the images are taken while one and the same light source is used, and the light source provides pulsed illumination, the images will be naturally synchronized by this light source.

In FIG. 5B, the two image capturing units image one and the same organism at locations 122 and 124. The imaging system typically works in video mode, such that all passing objects are recorded. If two objects pass one after the other, the images associated with each one will be analyzed separately. It is also possible the two objects are so close to one another that it is not feasible to separate them downstream and so they may both be discarded. Preferably, the organisms are conveyed one by one with a space there-between. The safe space can be measured in units of time. In the proposed embodiment, it is ˜0.5 sec or more between successive larvae. Unsorted larvae may be fed into the flow system through an entrance container, like in FIG. 1. The larvae in the container may be gently mixed, to overcome their self-movement and prevent clustering, through bubbling of air. The air may also be used to pressurize the container and drive the larvae through its outlet and into the flow system. Once a larva exits the entrance container into the flow system, it is detected by a sensor (e.g., photodiode). This triggers the system to switch a valve, such that (1) no more larvae (or water) can flow out of the entrance container, and (2) water flows into the system from a second, different container which contains only water. (No containers and valves are shown in this drawing). The water flow from the second container drives the larva that entered the system through the flow system and passes the detection region. Once the system is ready to accept a new larva, the valve is switched again so that (1) water and larvae can flow again out of the entrance container, and (2) no water can flow from the second container.

In different embodiments, imaging of the pre-adult insects, shrimp and fish in the flow-channel can be done from N>1 different directions, while using M image capturing units, with M<N.

FIG. 6 depicts one such embodiment 28.4 of the electro-optical module, where a single image capturing unit (108) images the pre-adult insects, or shrimp or fish in any stage from two different directions [shown from top-view]. Whereas, the first direction faces the sensor of the image capturing unit, the second direction is at an angle to it and the light is guided to the sensor by a setup of mirrors (126, 128) and a beam splitter or dichroic mirror (130). The optical path from the organism to the sensor will not necessarily be the same along the two directions. This may be corrected with an optical element (e.g., retarder) placed along the shorter path (132) to ensure that the images associated with the different directions are all in focus. Note that in such a setup, the images from the different directions will be formed simultaneously on the image capturing unit sensor. Also note that in order to avoid significant image data overlap from the two directions, each image will be formed on a different region of the sensor (e.g., by adjusting the angle of the beam splitter or dichroic mirror (130). Alternatively, the different illumination sources associated with different imaging directions captured on the same image capturing sensor, may be toggled on and off such that for any given image, only one illumination source will be switched on.

If different cameras are imaging the same region of interest and associated with different light sources, then the corresponding light pulses should be synchronized.

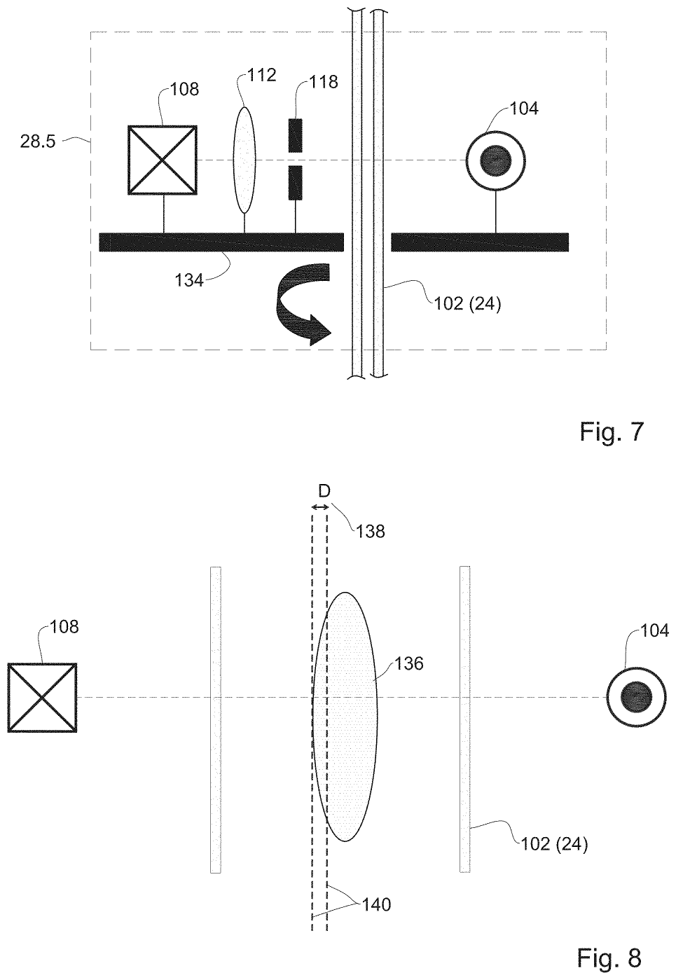

FIG. 7 depicts yet another embodiment where the number of imaging directions is bigger than the number of image capturing units [shown from side-view]. Theoretically, the number of imaging directions may be more than four.

In the embodiment of FIG. 7, the flow-channel is stationary, whereas the electro-optical unit 28.5 with the illumination and imaging elements, is rotatable about the flow channel.

In the example shown, a single image capturing unit together with its focusing optics, blocking element and illumination source are all attached to a rotating stage (134). The rotation itself may either be continuous or back-and-forth. The former option is less strenuous on the motor, but requires the use of a slip-ring or the like if any wires are attached to the rotating system (e.g., electronic, data) to prevent their twisting. This problem is also circumvented if the latter option of back-and-forth rotation is used.

FIG. 8 illustrates how the imaging units may operate according to one option of the proposed imaging technology. The figure shows a portion of the electro-optical module at the detection region. A transparent organism (say, larva 136) flowing via a fluidic channel 102 (24) is illuminated by while light from light source 104. The imaging unit or sensor 108 (say, an RGB camera) images the larva using the depth-of-field D. Dash lines 140 indicate the borders of D, within which sex organs of the larva are expected to be found. The organism 136 is imaged from different directions. Here, for simplicity, only one direction is shown using camera 108, with the extended depth of field D (138). In this example, D spans one-fourth of the organism thickness and is marked as the region between the dashed lines (140).

According to one embodiment the present invention provides a system for sex separation of organisms such as insects fish and shrimp, comprising. (a) a fluidic channel having an inner space and an outer wall, the fluidic channel comprising an inlet and at least one outlet, wherein the channel is configured to allow flow of the organisms suspended in liquid media; (b) a processor; and, (c) a controller in-communication with the processor; wherein the fluidic channel comprises a classification region, and the classification region comprises an electro-optical module in-communication with the processor, the electro-optical module comprises at least one sensor configured to acquire optical data of an individual organism and to transmit the optical data to the processor, further wherein the processor is configured to process the acquired optical data, to classify the organism based on the acquired optical data, and to instruct the controller to sort the organism based on the classification of the organism.

According to a further embodiment of the system of the present invention, the inner space of the fluidic channel is adjustable to the dimensions of a single organism.

According to a further embodiment of the system of the present invention, the fluidic channel is designed to allow passage of a single organism at a time.

According to a further embodiment the system is further configured to control movement of the organism when passing through the fluidic channel.

According to a further embodiment of the system of the present invention, the flow of the organism suspended in the liquid media through the fluidic channel is a passive flow.

According to a further embodiment of the system of the present invention, the flow of the fish suspended in the liquid media through the fluidic channel is an active flow.

According to a further embodiment of the system of the present invention, the flow is driven by a pump, vacuum, pressurizing means such as pressurized gas, gravity forces or any combination thereof.

According to a further embodiment the system comprises more than one outlet, wherein each outlet is independently operated by the controller.

According to a further embodiment of the system of the present invention, each of the one or more outlets is associated with a classification category selected from the group consisting of male, female and other.

According to a further embodiment of the system of the present invention, the at least one outlet having an “open” and a “closed” configuration.

According to a further embodiment of the system of the present invention, the outlet is operably coupled to a container.

According to a further embodiment of the system of the present invention, the fluidic channel further comprises a separation region, wherein the separation region is located between the detection region and the at least one outlet; and wherein the separation region comprises at least one guiding tool configured to guide the organism to an outlet associated with the classification.

According to a further embodiment of the system of the present invention, the guiding tool is selected from the group consisting of one or more valves, a transducer configured to generate acoustic radiation forces, electrodes configured to apply forces to the liquid media, electrodes configured to apply dielectrophoretic forces to the organism, controlled, liquid flow from one more additional directions and any combination thereof.

According to a further embodiment of the system of the present invention, the fluidic channel further comprises a destruction region; wherein the destruction region comprises at least one destroying tool in-communication with the controller; and wherein the destroying region has an “on” and “off” configurations operated by the controller.

According to a further embodiment of the system of the present invention, the at least one destroying tool is selected from the group consisting of a laser beam, a high-power electric field, ultrasonic blasts, a grinding tool, a squashing tool and any combination thereof.

According to a further embodiment of the system of the present invention, the controller is configured to execute functions selected from the group consisting of: receiving instructions from the processor, controlling flow rate, switching on the guiding tool, switching off the guiding tool, switching on the destroying tool, switching off the destroying tool, switching configuration of the outlet from “open” to “close”, and, switching configuration of the outlet from “closed” to “open”.

According to a further embodiment of the system of the present invention, the sensor is selected from the group consisting of an optical detector, a camera, a photodiode, a photomultiplier, an image acquisition sensor, an optical acquisition sensor, an electro-optical sensor, light detector, a photon sensor, a reflectometer, a photodetector, a spectral image sensor, and any combination thereof.

According to a further embodiment of the system of the present invention, the sensor is an imaging sensor selected from the group of consisting of RGB frequency spectrum, broad spectrum, hyperspectral, visible light frequency range, near infrared (NIR) frequency range, infrared (IR) frequency range, monochrome, specific light wavelengths (e.g., LED and/or laser and/or halogen and/or xenon and/or fluorescent), UV frequency range and any combination thereof.

According to a further embodiment of the system of the present invention, the electro-optical module of the classification region comprises at least one of: one or more light sources configured to illuminate the classification region, one or more image sensors, one or more acoustic sources configured to generate sound waves that pass through the classification region, at least one optical element and an internal control unit in-communication with the processor. According to a further embodiment of the system of the present invention, the electro-optical module comprises more than one light source, wherein each light source is configured to emit light with predetermined spectrum and/or intensity.

According to a further embodiment of the system of the present invention, the electro-optical module is configured to obtain multiple images of the organism from different angles.

According to a further embodiment of the system of the present invention, the image acquisition sensor is a high-frame rate.

According to a further embodiment of the system of the present invention, the light source illumination is stroboscopic or pulsed.

According to a further embodiment of the system of the present invention, the optical element is selected from the group consisting of a lens, a mirror, a polarizer, an excitation filter, an emission filter, a dichroic mirror, an optical coating such as antireflective coating, an optical grating and any combination thereof.

According to a further embodiment of the system of the present invention, the optical element comprises at least one stereoscopic microscope, at least one fluorescence microscope, or a combination thereof.

According to a further embodiment of the system of the present invention, the sensor is attached to the at least one microscope.

According to a further embodiment, the system of the present invention further comprises a sensor in-communication with the controller, wherein the controller is further configured to control the number of individual organisms at the entrance to the fluidic channel based on the data acquired by the sensor, to thereby allow passage of single organism at a time to the fluidic channel.

According to a further embodiment of the system of the present invention, the processor is configured to perform functions selected from receiving the optical data of an individual organism acquired by the sensor of the electro-optical module, processing the optical data, extracting a set of parameters indicative of the sex of the organism from the optical data, classifying the organism based on the set of parameters extracted from the optical data, providing instructions to the controller based on the classification of the organism.

According to a further embodiment of the system of the present invention, the processor is configured to classify the individual organism into at least two classes selected from the group consisting of male and non-male.

According to a further embodiment of the system of the present invention, the processor is configured to classify the individual fish into at least two classes selected from the group consisting of female and non-female.

According to a further embodiment of the system of the present invention, the processor is configured to classify the individual fish into three classes selected from the group consisting of male, female, and other.

According to a further embodiment of the system of the present invention, the features of the organism including the features (parameters) indicative of the sex of the organism are selected from at least one of a first, a second and a third group respectively comprising the following:
the first group: overall size of the organism, morphology of the organism, shape, segment size ratio,
the second group of color-related features comprising: absorption, transmission, IR (Infrared) absorption, IR transmission, color, fluorescence,
the third group: gonad disc morphology, secondary sex organ morphology, gonad size, gonad morphology, gonad autofluorescence, size of testes, size of male accessory glands, morphology of testes, morphology of male accessory glands, autofluorescence of testes, autofluorescence of male accessory glands, size of the developing male and female primary or secondary reproductive structures, primitive sex organs, morphology of the developing male and female primary or secondary reproductive structures, autofluorescence of the developing male and female primary or secondary reproductive structures and any combination thereof.

According to a further embodiment of the system of the present invention, the detection/classification region further comprises a glass capillary through which the organism flows.

According to a further embodiment, the system of the present invention further comprises a thermostat optionally operated by the controller, wherein the thermostat is configured to control the temperature in the fluidic channel.

According to a further embodiment of the system of the present invention, the liquid media is water or isotonic aqueous solution.

According to a further embodiment of the system of the present invention, the sorting rate is about 0.1 to 100 organisms per second.

According to a further embodiment of the system of the present invention, the inner space of the fluidic channel has the size in the range of 0.1 mm to 10 mm.

According to a further embodiment of the system of the present invention, the sorting accuracy is in the range of 50% to 100%, particularly 80% to 100%, more particularly at least 90%.

According to a further embodiment of the system of the present invention, the sorting specificity is in the range of 50% to 100%, particularly 80% to 100%, more particularly at least 90%.

According to a further embodiment of the system of the present invention, the sorting selectivity is in the range of 50% to 100%, particularly 80% to 100%, more particularly at least 90%.

According to a further embodiment of the system of the present invention, the organism is a pre-adult insect or shrimp or fish larva or fish fry or fish fingerling or adult fish.

According to a further embodiment, the system of the present invention further comprises a container in fluid connection with the fluidic channel, the container contains unsorted organisms suspended in a liquid media, the container has an outlet configured to allow passage of a single organism at a time to the fluidic channel, the container further comprises pressurizing means, such as pressurized gas, vacuum, gravity forces and/or one or more pumps to drive a flow of the unsorted organisms towards the outlet of the container to the fluidic channel.

It is further within the scope of the present invention to provide a container in fluid connection with fluidic channel for sex separation of organisms, the container contains unsorted organism suspended in a liquid media, the container has an outlet configured to allow passage of a single organism at a time to the fluidic channel, the container further comprises pressurizing means, such as pressurized gas, vacuum, gravity forces and/or one or more pumps to drive a flow of unsorted organisms towards the outlet of the container to the fluidic channel.

It is further within the scope of the present invention to provide a computer implemented method of sex separation of organisms such as pre-adult insects, fish or shrimp, the method comprising: (a) providing the system for sex separation of organism as defined in any of the above; (b) streaming the organism suspended in the liquid media through the inlet into the fluidic channel towards the classification region; (c) acquiring optical data of the individual organism at the classification region by one or more sensors of the electro-optical module; (d) transmitting the optical data of the individual organism to the processor; (e) processing the optical data and classifying the individual organism based on a set of parameters indicative of the sex of the organism extracted from the optical data: (f) providing instructions to the controller based on the classification of the individual organism; and, (g) sorting the organism according to the instructions received by the controller.

It is further within the scope of the present invention to provide the method as defined above, wherein the step of sorting of the organisms according to the instructions received by the controller comprises at least one of: controlling flow rate, controlling sorting rate, switching on the guiding tool, switching off the guiding tool, switching on the destroying tool, switching off the destroying tool, switching the configuration of the outlet from “open” to “closed”, and switching the configuration of the outlet from “closed” to “open”.

It is further within the scope of the present invention to provide the method as defined in any of the above, wherein the organisms are classified into two classes, wherein the two classes are male and non-male.

It is further within the scope of the present invention to provide the method as defined in any of the above, wherein the organisms are classified into two classes, wherein the two classes are female and non-female.

It is further within the scope of the present invention to provide the method as defined in any of the above, wherein the organisms are classified into three classes, wherein the three classes are male, female and other.

It is further within the scope of the present invention to provide the method as defined in any of the above, wherein the individuals classified as non-male are directed to the destruction region to be destroyed by the at least one destroying tool.

It is further within the scope of the present invention to provide the method as defined in any of the above, wherein the individuals classified as non-female are directed to the destruction region to be destroyed by the at least one destroying tool.

It is further within the scope of the present invention to provide the method as defined in any of the above, wherein the organism is a pre-adult insect or shrimp or fish in any stage (whether larva, fry, fingerling or an adult).

It is further within the scope of the present invention to provide the method as defined in any of the above, further comprises steps of adjusting the dimensions of the inner space of the fluidic channel to allow passage of a single organism at a time.

It is further within the scope of the present invention to provide the method as defined in any of the above, further comprises steps of adjusting the dimensions of the innerspace of the fluidic channel such that a single organism extends parallel to the channel walls.

It is further within the scope of the present invention to provide the method as defined in any of the above, further comprises steps of pressurizing or directing by gravity forces unsorted organisms towards the outlet of the fluidic channel.

It is further within the scope of the present invention to provide the method as defined in any of the above, further comprises steps of reducing or elevating the temperature of the liquid media so as to control wriggling movements of the organism flowing within the fluidic channel.

It is further within the scope of the present invention to provide the method as defined in any of the above, further comprises steps of reducing the temperature of the liquid media so as to reduce wriggling movements of the organism flowing within the fluidic channel.

It is further within the scope of the present invention to provide the method as defined in any of the above, comprising steps of acquiring optical data by a sensor selected from the group consisting of an optical detector, a camera, a photodiode, a photomultiplier, an image acquisition sensor, an electro-optical sensor, an optical acquisition sensor, a light detector, a photon sensor, a reflectometer, a photodetector, a spectral image sensor, and any combination thereof.

It is further within the scope of the present invention to provide the method as defined in any of the above, wherein the electro-optical module of the classification region comprises at least one of: one or more light sources configured to illuminate the classification region, one or more acoustic sources configured to generate sound waves that pass through the classification region, one or more image sensors, at least one optical element and an internal control unit in-communication with the processor.

Additionally, it is within the scope of the present invention to provide the method as defined in any of the above, wherein the classifying comprises using a trained neural network.

It is further within the scope of the present invention to provide the method as defined in any of the above, wherein the step of processing comprises steps of analyzing the optical data using computer implemented algorithm trained to generate output based on the optical data.

It is further within the scope of the present invention to provide the method as defined in any of the above, wherein the computer implemented algorithm is trained to generate output based on predetermined feature vectors or attributes extracted from the optical data.

It is further within the scope of the present invention to provide the method as defined in any of the above, wherein the method comprises steps of implementing with the algorithm a training process according to a training dataset comprising a plurality of training images of a plurality of organisms captured by the at least one imaging sensor, wherein each respective training image of the plurality of training images is associated with the sex determination of the organism depicted in the respective training image.

It is further within the scope of the present invention to provide the method as defined in any of the above, wherein the training process comprises steps of (a) capturing images of organism using an imaging sensor; (b) classifying images into classification categories by applying a tag associated with parameters or attributes indicative of the sex of the organism extracted from the optical data; and (c) applying a computer vision algorithm to determine a set of feature vectors associated with each classification category.

It is further within the scope of the present invention to provide the method as defined in any of the above, further comprising steps of applying a machine learning process with the computer implemented trained algorithm to determine the sex of the imaged organism.

It is further within the scope of the present invention to provide the method as defined in any of the above, wherein the algorithm is implemented with a machine learning process using a neural network with the processed data.

It is further within the scope of the present invention to provide the method as defined in any of the above, wherein the machine learning process comprises computing by the at least one neural network, a tag of at least one classification category for the at least one organism, wherein the tag of at least one classification category is computed at least according to weights of the at least one neural network, wherein the at least one neural network is trained according to a training dataset comprising a plurality of training images of a plurality of organisms captured by the at least one imaging sensor, wherein each respective training image of the plurality of training images is associated with the tag of at least one classification category of at least one organism depicted in the respective training image; and generating according to the tag of at least one classification category, instructions for execution by the controller.

It is further within the scope of the present invention to provide the method as defined in any of the above, wherein the sensor is configured for image capturing and processing, with or without using Artificial Intelligence (AI) and/or machine learning and/or neural networks.

It is further within the scope of the present invention to provide a computer implemented algorithm comprising code to perform the steps of the method as defined in any of the above.

According to a further embodiment, the computer implemented algorithm may be a machine learning algorithm. In still a further embodiment, the machine learning algorithm may use verified training data.

In other words, there is provided a software product comprising computer-implementable instructions and data stored on a non-transitory computer readable storage medium and designed to cause performing steps of the method as described in any of the described embodiments.

There is also provided a non-transitory computer readable storage medium, accommodating the software product stored thereon.

In order to understand the invention and to see how it may be implemented in practice, some exemplary embodiments will now be described, by way of non-limiting examples only.

### Example 1: A System for Sex-Sorting of Fish

An exemplified sex-sorting system essentially is based on the following: fish larvae or fries or fingerlings or adults are kept in their natural environment, namely, water. Unsorted fish flow, under pressure or gravity, towards the outlets of the system. The inner dimensions of the flow channel were chosen such that only a single fish can pass at a time. Furthermore, these dimensions constrict the fish to extend parallel to the channel walls. The temperature of the liquid may also be cooled so as to reduce wriggling movements. As the fish pass through a classification region, they are imaged.

Additional Optional Features:

Aqueous medium used to flow the fish (typically water) may be cooled in order to reduce their movements during the sorting process.

The unsorted fish may be kept dispersed in the liquid before entering the sorting system through agitation. This may help fish enter the system one by one and at a constant rate.

A sensor at the entrance to the system may be used to detect if two or more fish have entered it at close proximity. If this occurs, the larvae are diverted outside to prevent possible mistakes.

Pressurized gas may be used to mix the fish and drive them through the system.

Imaging with a color camera enables to improve differentiation by using the different absorption characteristics of the different organs.

Imaging from two or more distinct angles (observation directions) in order to improve detection of objects of interest (e.g., gonads should be visible from one of the angles). This may be done using two or more cameras or by using a single camera and directing the light from different regions on different parts of the imaging sensor.

High frame rate (>30 frames per second) and stroboscopic illumination may be used in order to image the flowing larvae without having to stop them before capturing the image.

In the imaging region/unit, the fish may flow through a glass capillary to improve image quality. A square or rectangular capillary may be used, for a more uniform background and to remove spherical aberrations.

Both male and female primary and secondary primitive (not fully developed) sex organs can be used.

With respect to the imaging platform, image quality was compared between an inverted Leica DM IRE2 microscope (×10, NA 0.3 objective), an Olympus SZH10 stereo microscope and a Raspberry Pi with V1 camera module and macro lens. No significant differences were found between the three systems, and the gonads, when visible, were clearly discernible in all three.

The following operations were observed:

The system receives a container with water and unsorted larvae,

The larvae pass one-by-one into a flow system,

The flowing larvae are imaged,

After imaging, the system switches the larvae to appropriate outlets (male and female containers) according to a predesigned computer algorithm,

The larvae are not harmed by the process.

It can be concluded by the experimental results that:

No damage was done to the larvae that were sex sorted by the system of the present invention and all of the larvae developed properly to adulthood.

### Example 2: Sorting Criteria

In some embodiments of the technique, the viability of an organism is determined by its light absorption pattern, i.e., its general opacity and the level of uniformity in light absorption. In most cases, but not exclusively, live organisms are less opaque (less absorptive) and less uniform in their absorption profile than dead ones.

In other embodiments, the growth potential of an organism is determined by a combination of its overall size and shape (e.g., width to length ratio) and/or number of specific structures like head and mouth parts.

In further embodiments, the health of an organism is determined by its color under a broad spectrum illumination. The observed color and color ratios of the organism are partly the result of light refraction characteristics of its exterior and/or morphological signals such as the presence or absence of structures caused by disease or the organism's response to a pathogen, or a combination thereof.

In embodiments where sex is being sorted, the determining factor may be the existence or shape of parts of the reproductive system. For example, the existence of secondary male glands in the ninth (anal) segment of mosquito larvae correlates with male outcome.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove.

Features described for a method apply mutatis mutandis to the system corresponding to the method, and vice versa. Additional features described for the method of determining features, structures and/or organs in the organisms of interest are also applicable to the method of sorting such organisms, and vice versa. Additional features described for the system for determining features, structures and/or organs in the organisms are also applicable to the system for sorting the organisms, and vice versa. Only part of possible combinations have been claimed in the present set of claims at this stage.

The scope of the present invention is generally defined by the appended claims and includes some of combinations and sub-combinations of the various features described hereinabove, as well as some variations and modifications thereof, which would occur to persons skilled in the art upon reading the foregoing description. More combinations and sub-combinations may be constructed based on the description and may be brought to the claims at a later stage.

1. A computer implemented method for automated detection of features, structures and/or organs in organisms being shrimp, fish or pre-adult insects, the method comprising:
a) providing a flow-system comprising a fluidic channel with an inlet, a detection region and at least one outlet, an electro-optical module for monitoring the detection region and a processor;
b) ensuring passage of the organisms via a fluidic channel towards the detection region,
c) imaging an individual organism in the detection region by one or more sensors of the electro-optical module, thereby acquiring optical data on the organism,
d) transmitting the acquired optical data to the processor,
e) analyzing the obtained optical data by the processor, for detecting presence of one or more of said features, structures and/or organs of interest.

2. The method of claim 1, performing the imaging of the individual organism in the detection region with an extended depth of field that spans at least one-fourth of the organism's thickness, from at least two different observation directions.

3. The method of claim 1, further performing sorting of the organisms by the following steps:
performing classification of the individual organism, using the acquired optical data, wherein said classification being based on one or more morphologic features and/or one or more color-related features and/or one or more sex-related features, and
sorting said organism based on its classification.

4. A system for automated detection of one or more features, structures and/or organs in organisms being shrimp, fish or pre-adult insects, the system comprising:
a) a fluidic channel comprising an inlet, a detection region and at least one outlet, said channel is configured to allow flowing there-though of the organisms suspended in liquid media;
b) an electro-optical module for monitoring the detection region,
c) a processor in communication with the electro-optical module,
wherein
said electro-optical module comprises at least one sensor and is configured to acquire optical data by imaging an individual organism, to transmit said optical data to the processor, and
the processor is configured to receive, process and analyze the optical data acquired by the electro-optical module so as to detect said features, structures and/or organs of interest in the organism.

5. The system of claim 4, wherein the electro-optical module is configured for capturing images of an individual organism in the detection region from at least two different observation directions and with an extended depth of field that spans at least one-fourth of the organism's thickness.

6. The system of claim 4, configured for sorting of said organisms, further comprising a controller in-communication with said processor, wherein
the processor being further configured to classify the organism based on the analysis results, on one or more morphologic features and/or one or more color-related features and/or one or more sex-related features, and to instruct the controller to sort said organism based on the classification.

7. The method of claim 1 wherein said pre-adult insects are in the shape of a larva or a pupa or a nymph; said shrimp are in the shape of a larva or a post-larva or a juvenile; and said fish are in the shape of a larva or a fry or a fingerling or an adult.

8. The method of claim 1, wherein the organisms at the detection region are illuminated by pulsed or strobed illumination.

9. The method as claimed in claim 8, wherein the pulse duration is less than 50 microseconds.

10. The method as claimed in claim 1, performed with broad spectrum illumination at the detection region.

11. The method as claimed in claim 1, wherein the organisms flow through the fluidic channel one at a time.

12. The method as claimed in claim 2, wherein the angle between said two observation directions is of about 90°.

13. The method as claimed in claim 1 wherein the imaging of the individual organism is taken from different locations along the fluidic channel at the detection region.

14. The method as claimed in claim 1, wherein the optical data not containing the organisms of interest is used for adjusting and pre-processing the optical data containing said organisms.

15. The method of claim 1, wherein the organisms are classified at least into two classes, wherein said two classes being selected from at least the following pairs: male and non-male, female and non-female, male and female, alive and dead, healthy and sick, strong and weak, quickly developing or retarded.

16. The method of claim 1 further comprising steps of reducing or elevating the temperature of the liquid media so as to control wriggling movements of the organisms flowing within the fluidic channel.

17. The method of claim 1, wherein said steps of detecting and/or sorting comprise using a trained neural network.

18. The method as claimed in claim 1, wherein the imaging provides smearing of the obtained images by less than 0.25% of the full frame.

19. The method as claimed in claim 1, wherein said features for analyzing or sorting the organisms include one or more features from at least one of the following non-exhaustive groups:
a first group of morphologic features comprising: overall size of the organism, morphology of the organism, shape, segment size ratio,
a second group of color-related features comprising: absorption, transmission, IR (Infrared) absorption, IR transmission, color, fluorescence,
a third group of sex-related features comprising: gonad disc morphology, secondary sex organ morphology, gonad size; gonad morphology, gonad autofluorescence, size of testes, size of male accessory glands, morphology of testes, morphology of male accessory glands, autofluorescence of testes, autofluorescence of male accessory glands, size of the developing male and female primary or secondary reproductive structures, primitive sex organs, morphology of the developing male and female primary or secondary reproductive structures, autofluorescence of the developing male and female primary or secondary reproductive structures and any combination thereof.

20. The system as claimed in claim 4, wherein the electro-optical module comprises at least one of; one or more light sources configured to illuminate the detection region, one or more acoustic sources configured to generate sound waves that pass through the detection region, one or more image sensors, at least one optical element and an internal control unit in-communication with the processor.

21. The system as claimed in claim 4, comprising at least one blocking structure associated with a suitable sensor, such that only light from the detection region can reach it.

22. The system as claimed in claim 6, wherein the fluidic channel further comprises a separation region, wherein said separation region is located between the detection region and the at least one outlet; and wherein said separation region comprises at least one guiding tool controlled by the controller and configured to guide said organism to an outlet associated with the organism's classification.

23. The system as claimed in claim 4, wherein the fluidic channel further comprises a destruction region; wherein said destruction region comprises at least one destroying tool in-communication with the controller; and wherein said destroying region has “on” and “off” configurations operated by the controller.

24. The system as claimed in claim 4, further comprising an additional sensor in-communication with the controller, wherein the controller is further configured to control the amount of organisms at the entrance to the fluidic channel based on the data acquired by said sensor, to thereby allow passage of single organism at a time to the fluidic channel.

25. The system as claimed in claim 4, configured to provide illumination pulses with duration substantially close to that determined in the following equation:
dT=(N*p*PS)/(v*M) (1)
where dT is the pulse duration,
N is the size of the organism of interest in the flow direction,
p is the % of allowed smearing for the object of interest,
PS is the pixel size of the imaging sensor,
v is the flow velocity of the organism of interest in the channel,
M is the optical magnification of the imaging system.

26. The system as claimed in claim 4, wherein the fluidic channel is a transparent capillary having a square or rectangular cross-section.
